(19) United States
(12) Plant Patent Application Publication
(10) Pub. No.: US 2002/0152507 P1
(43) Pub. Date: Oct. 17, 2002

(54) SHRUB ROSE VARIETY 'POULATTRA'

(75) Inventors: L. Pernille Olesen, Fredensborg (DK); Mogens S. Olesen, Fredensborg (DK)

Correspondence Address: Poulsen Roser Pacific, Inc., 2550 Old Military Road, Central Point, OR, 97502, US

(73) Assignee: Poulsen Roser ApS.

(21) Appl. No.: 09/776,563

(22) Filed: Feb. 1, 2001

(51) Int. Cl. A01H 5/00

(52) U.S. Cl. PLT/108

(57) ABSTRACT

A new shrub rose which has abundant, dark red flowers and attractive foliage. This new and distinct variety has shown to be uniform and stable in the resulting generations from asexual propagation.

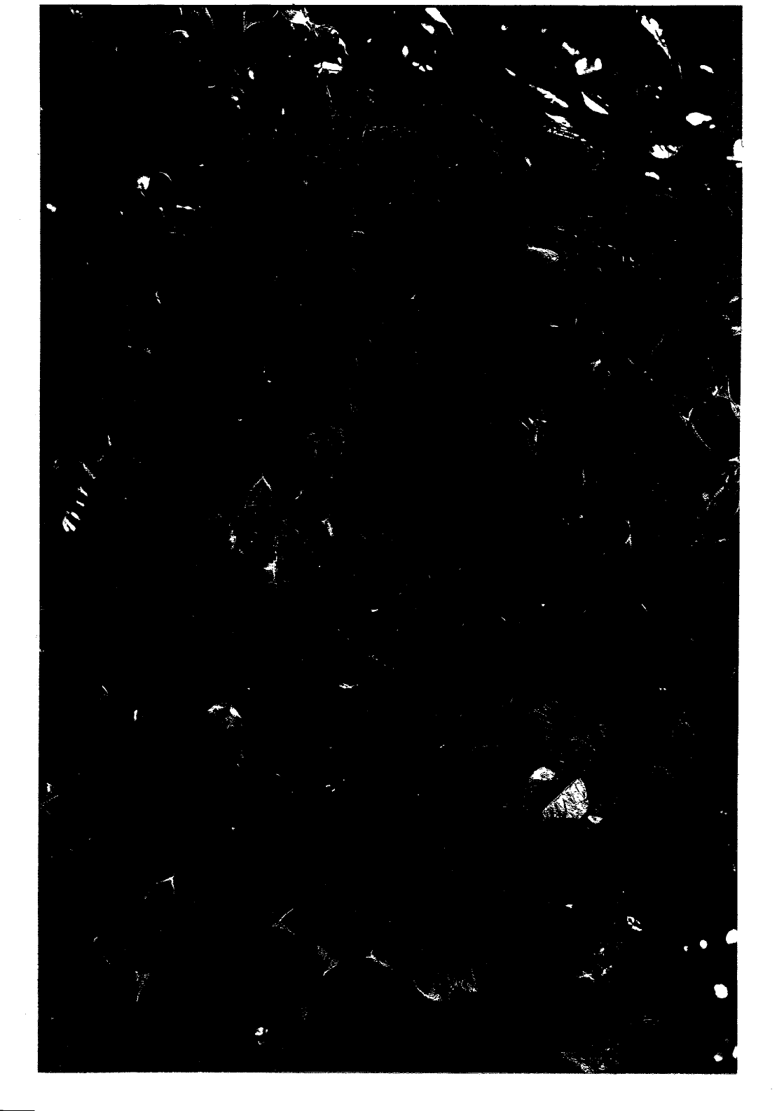

### SUMMARY OF THE INVENTION

The present invention constitutes a new and distinct variety of shrub rose which originated from a controlled crossing between ‘Kormixal Suffolk’ and ‘an unnamed seedling’, both unpatented varieties. The two parents were crossed during the summer of 1993 and the resulting seeds were planted in a controlled environment in Fredensborg, Denmark. The new variety is named ‘POULattra’.

The new rose may be distinguished from its seed parent, ‘Kormixal Suffolk’, by the following combination of characteristics:

1. ‘POULattra’ exhibits dark red blooms, whereas ‘Kormixal Suffolk’ exhibits lighter red blooms with a yellow center.

2. ‘POULattra’ has small double blooms, whereas the seed parent has semi-double blooms.

The new variety may be distinguished from its pollen parent, ‘an unnamed seedling’ by the following combination of characteristics:

1. ‘POULattra’ is a shrub rose with a spreading, bushy habit, whereas the pollen parent is a miniature rose.

2. ‘POULattra’ has larger leaves and more foliage than the pollen parent.

The objective of the hybridization of this rose variety was to create a new and distinct variety for garden use with unique qualities, such as:

1. Uniform and abundant dark red flowers;

2. Vigorous, but compact growth when propagated both as a budded rose and on its own roots;

3. Disease resistance.

This combination of qualities is not present in previously available commercial cultivars of this type and distinguish ‘POULattra’ from all other varieties of which we are aware.

As part of their rose development program, L. Pernille Olesen and Mogens N. Olesen germinated the seeds from the aforementioned hybridization during winter 1993 and conducted evaluations on the resulting seedlings in a controlled environment in Fredensborg, Denmark.

‘POULattra’ was selected in the spring 1994 by the inventors as a single plant from the progeny of the aforementioned hybridization.

Asexual reproduction of ‘POULattra’ by traditional budding and rooted cuttings was first done by L. Pernille and Mogens N. Olesen in their nursery in Fredensborg, Denmark in summer 1994. This initial and other subsequent asexual propagations conducted in controlled environments have demonstrated that the characteristics of ‘POULattra’ are true to type and are transmitted from one generation to the next.

### BRIEF DESCRIPTION OF THE DRAWING

The accompanying color illustration shows as true as is reasonably possible to obtain in color photographs of this type the variety rose ‘POULattra’. Specifically illustrated in SHEET 1 are ‘POULattra's’ foliage, flower buds, partially open buds, and open blooms.

### DETAILED DESCRIPTION OF THE VARIETY

The following is a description of ‘POULattra’, as observed in its growth during trials at the testing station Prüfstelle Rethmar under the testing authority Bundessortenamtin in Hannover, Germany and at the inventor's nursery in Fredensborg, Denmark. Color references are made using The Royal Horticultural Society (London, England) Colour Chart, 1995, except where common terms of color are used.

For a comparison, several physical characteristics of the rose variety ‘POULria’, a rose variety from the same inventors described and illustrated in U.S. Plant patent application Ser. No. 09/274,686 dated Mar. 24, 1999 are compared to ‘POULattra’ in Chart 1.

CHART 1

| | ’POULattra' | ’POULria' |
|---|---|---|
| Color of open | Red Group 53D | Red Group 53A to |
| flower, outer | | Red-Purple Group |
| side, middle | | 59C. |
| zone. | | |
| Petalage | 20-25 | 40-50 |
| Upon opening, | Yellow Group 4D | White Group 155C |
| color basal | | |
| petal spot, | | |
| outer side | | |

Parents:

Seed Parent.—‘Kormixal Suffolk’.

Pollen Parent.—‘Unnamed seedling’.

Classification:

Botanical.—Rosa hybrida.

Commercial.—Shrub rose.

### Flower and Flower Bud

Blooming habit: Continuous.

Flower bud:

Bud form.—Ovate.

Sepals.—Weak appendages.

Receptacle.—Size: Small.

Peduncle.—Surface: Above average abundance of hairs and prickles.

Borne.—From top of plant. Between 5 to 40 flowers per branch.

Flower bloom:

Fragrance.—Slight spicy scent.

Size.—Small. Average flower diameter is 40-60 mm when open.

Form.—Shape of flower when viewed from the side: Upper part: Flat. Lower part: Flat. Viewed from above: Irregularly rounded.

Petalage.—Double. Average range: 20-25 petals under normal conditions.

Color:

Petals.—Inner Side: Middle zone: Red Group 53A. Marginal zone: Red Group 53A. Basal petal spot: Yellow Group 4D. Outer side: Middle zone: Red Group 53D. Marginal zone: Red Group 53D. Basal petal spot: Yellow Group 4D.

General Tonality: On open flower Red Group 53D. No change in the general tonality.

Petals:

Petal Reflex.—Weak to medium.

Undulation of Margin.—Weak.

Size.—Small.

Quantity.—Few.

Reproductive Organs:

Filaments.—Color: Yellow.

Hips.—Shape: Pitcher-shaped.

### Plant

Plant growth: Low, compact, bushy to slightly spreading, even growth. When grown as a budded field grown plant on Rosa multiflora understock, the average height of the plant is 40-60 cm and the average width is 50 cm.

Stems:

Thorns.—Incidence: Few. Size: Small. Shape: Concave.

Plant foliage:

Leaf size.—Small.

Abundance.—Average.

Glossy.—Upper side average glossiness.

Color.—Upper Leaf Surface: Dark green with reddish edges. Juvenile foliage: Exhibits anthocyanin on young shoots. Anthocyanin intonation: Color: Bronze to reddish brown.

Plant leaves and leaflets:

Leaflet.—Cross Section: Flat. Margin Undulation: Absent to very weak.

Terminal Leaflet.—Length of blade: Short. Width of blade: Narrow. Shape: Rounded.

Disease resistance: Above average resistance to mildew, rust, black spot, and Botrytis under normal growing conditions in Fredensborg, Denmark and Hannover, Germany.

Cold Hardiness: The variety ‘POULattra’ has been found to be cold hardy in Fredensborg, Denmark and Hannover, Germany.

1. A new and distinct variety of rose plant of the shrub rose class, substantially as herein illustrated and described as a distinct and novel rose variety due to its abundant dark red flowers, disease resistance, and extended period of bloom:
